(19) United States
(12) Patent Application Publication
(10) Pub. No.: US 2018/0274102 A1
(43) Pub. Date: Sep. 27, 2018

(54) METHOD OF FORMING METAL PATTERN

(71) Applicant: Kabushiki Kaisha Toshiba, Minato-ku (JP)

(72) Inventors: Yusuke TANAKA, Kawasaki (JP); Atsushi HIENO, Yokohama (JP); Tsutomu NAKANISHI, Yokohama (JP); Yasuhito YOSHIMIZU, Yokkaichi (JP); Akihiko HAPPOYA, Ome (JP)

(73) Assignee: Kabushiki Kaisha Toshiba, Minato-ku (JP)

(21) Appl. No.: 15/698,364

(22) Filed: Sep. 7, 2017

(30) Foreign Application Priority Data
Mar. 22, 2017 (JP) 2017-056485

(51) Int. Cl. C23C 18/18; C23C 18/16; C23C 18/34; C23C 18/40; C23F 1/00; C07D 251/12; H05K 3/18

(52) U.S. Cl. CPC C23C 18/1893; C23C 18/165; C23C 18/1608; C23C 18/1605; H05K 3/184; C23C 18/40; C23F 1/00; C07D 251/12; C23C 18/34

(57) ABSTRACT

A method of forming a metal pattern includes forming a catalyst adsorption layer by bringing a surface of a substrate into contact with a solution, the substrate having a base region and a plurality of protrusions provided on the base region, the base region includes a first material, the protrusions includes a second material different from the first material, the first and the second material being exposed on the surface, and the solution containing a compound having a triazine skeleton, a first functional group of any one of a silanol group and an alkoxysilyl group, and a second functional group of at least one selected from the group consisting of an amino group, a thiol group, and an azido group, forming a catalyst layer on the catalyst adsorption layer, forming a metal film on the catalyst layer by an electroless plating method, and removing the metal film on the protrusions.

### CROSS-REFERENCE TO RELATED APPLICATION

This application is based upon and claims the benefit of priority from Japanese Patent Application No. 2017-056485, filed on Mar. 22, 2017, the entire contents of which are incorporated herein by reference.

### FIELD

Embodiments described herein relate generally to a method of forming a metal pattern.

### BACKGROUND

In a semiconductor device, for example, a patterned metal film, that is, a metal pattern is used as a metal wiring layer or a hard mask for etching for forming a device structure. For the formation of the metal pattern, for example, an electroless plating method, which is high throughput and low cost and is capable of low temperature formation, is used.

Along with the scaling-down of a semiconductor device, the scaling-down of a metal pattern is also required. In the case of forming a fine metal pattern using an electroless plating method, it is desired that a conformal metal film can be formed on a substrate having a fine uneven pattern on its surface.

### BRIEF DESCRIPTION OF THE DRAWINGS

FIGS. 1A, 1B, 1C, 1D, and 1E are explanatory views of a method of forming a metal pattern according to a first embodiment;

FIGS. 2A, 2B, 2C, 2D, 2E, and 2F are explanatory views of a method for forming a metal pattern according to a second embodiment;

FIGS. 3A, 3B, 3C, 3D, 3E, 3F, and 3G are explanatory views of a method of forming a metal pattern according to a third embodiment;

FIGS. 4A, 4B, 4C, 4D, 4E, and 4F are explanatory views of a method of forming a metal pattern according to a fourth embodiment;

FIGS. 5A, 5B, 5C, 5D, and 5E are explanatory views of a method of forming a metal pattern according to a fifth embodiment;

FIGS. 6A and 6B are SEM photographs of Example 1;

FIG. 7 is a SEM photograph of Comparative Example;

FIG. 8 is a SEM photograph of Example 2;

FIG. 9 is a SEM photograph of Example 3;

FIG. 10 is a SEM photograph of Example 4;

FIGS. 11A and 11B are SEM photographs of Example 5; and

FIG. 12 is a SEM photograph of Example 6.

### DETAILED DESCRIPTION

A method of forming a metal pattern according to an embodiment includes: forming a catalyst adsorption layer by bringing a surface of a substrate into contact with a solution, the substrate having a base region and a plurality of protrusions provided on the base region, the base region including a first material, the protrusions including a second material different from the first material, the first material and the second material being exposed on the surface of the substrate, and the solution containing a compound having a triazine skeleton, a first functional group of any one of a silanol group and an alkoxysilyl group, and a second functional group of at least one selected from the group consisting of an amino group, a thiol group, and an azido group; forming a catalyst layer on the catalyst adsorption layer; forming a metal film on the catalyst layer by an electroless plating method; and removing the metal film on the protrusions.

Hereinafter, embodiments of the disclosure will be described with reference to the drawings. In the following description, the same or similar members and the like are denoted by the same reference numerals, and the description of the members and the like described once will be omitted as appropriate.

### First Embodiment

The method of forming a metal pattern according to this embodiment includes: forming a catalyst adsorption layer by bringing a surface of a substrate into contact with a solution, the substrate having a base region and a plurality of protrusions provided on the base region and containing a first material and a second material different from the first material, the first material and the second material being exposed on the surface of the substrate, and the solution containing a compound having a triazine skeleton, a first functional group of any one of a silanol group and an alkoxysilyl group, and a second functional group of at least one selected from the group consisting of an amino group, a thiol group, and an azido group; forming a catalyst layer on the catalyst adsorption layer; forming a metal film on the catalyst layer by an electroless plating method; and removing the metal film on the protrusions.

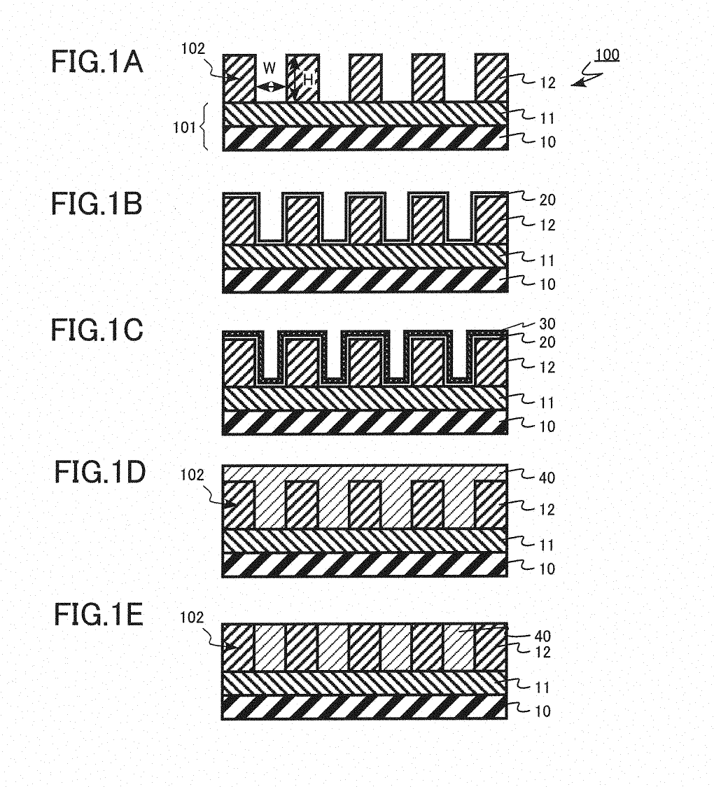

FIGS. 1A, 1B, 1C, 1D, and 1E are explanatory views of the method of forming a metal pattern according to this embodiment. FIGS. 1A, 1B, 1C, 1D, and 1E illustrate sectional views of a substrate on which a metal pattern is formed.

First, a substrate 100 is prepared (FIG. 1A). The substrate 100 is formed using a known process technology.

The substrate 100 has a base region 101 and a plurality of protrusions 102. The arrangement pitch of the protrusions 102 is, for example, 100 nm or less. Further, the ratio (H/W) of the height (H in FIG. 1A) of the protrusion 102 to the interval (W in FIG. 1A) between the protrusions 102 is, for example, 2 or more. The plurality of protrusions 102 serves as a guide pattern for forming a metal pattern.

The arrangement pitch of the protrusions 102, the interval between the protrusions 102, and the height of the protrusion 102 can be measured by observation with SEM (Scanning Electron Microscope).

Further, the substrate 100 has a first material and a second material different from the first material. The first material and the second material are exposed on the surface of the substrate 100.

The first material is an oxide, a nitride, or an oxynitride, and the second material is an oxide, a nitride, or an oxynitride different from that of the first material. The oxide is, for example, silicon oxide or aluminum oxide. The nitride is, for example, silicon nitride or aluminum nitride. The oxynitride is, for example, silicon oxynitride or aluminum oxynitride. Hereinafter, a case where the first material is silicon nitride and the second material is silicon oxide will be described as an example.

The base region 101 includes a silicon layer 10 and a silicon nitride layer 11 on the silicon layer 10. A silicon oxide layer 12 is provided on the silicon nitride layer 11. The silicon oxide layer 12 is patterned to form a plurality of protrusions 102. Silicon oxide and silicon nitride are exposed on the surface of the substrate 100.

Next, the surface of the substrate 100 is brought into contact with a solution containing a triazine compound having a triazine skeleton, a first functional group of any one of a silanol group and alkoxysilyl group, and a second functional group of at least one selected from the group consisting of an amino group, a thiol group, and an azido group, so as to form a catalyst adsorption layer 20 (FIG. 1B). The triazine compound of this embodiment is represented by Formula (1) below.

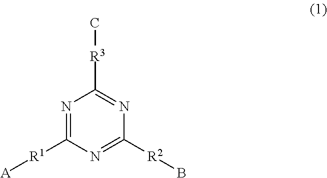

In Formula (1), at least one of A, B, and C is any one of a silanol group and an alkoxysilyl group, at least one of A, B, and C is at least one selected from the group consisting of an amino group, a thiol group, and an azido group, and R¹, R² and R³ are arbitrarily present linking groups.

Examples of the alkoxysilyl group include a trimethoxysilyl group, a dimethoxymethylsilyl group, a monomethoxydimethylsilyl group, a triethoxysilyl group, a diethoxymethylsilyl group, and a monoethoxydimethylsilyl group. For example, R¹, R², and R³ include a secondary amine or an alkyl chain. For example, R¹, R², and R³ do not exist, and an amino group, a thiol group, or an azido group may be bonded directly to a triazine ring.

For example, one of A, B and C is any one of a silanol group and an alkoxysilyl group, and the remaining two may be at least one selected from the group consisting of an amino group, a thiol group, and an azido group.

The solvent of the solution containing the triazine compound is, for example, water. The solvent of the solution containing the triazine compound is, for example, an alcoholic solvent such as methanol, ethanol, propanol, ethylene glycol, glycerin, or propylene glycol monoethyl ether.

The contact between the surface of the substrate 100 and the solution containing the triazine compound is performed, for example, by dipping the substrate 100 into the solution containing the triazine compound. Alternatively, the contact is performed by applying the solution containing the triazine compound onto the substrate 100.

The contact time of the surface of the substrate 100 and the solution containing the triazine compound is, for example, 1 minute or less.

Next, a catalyst layer 30 is formed on the catalyst adsorption layer 20. The catalyst layer 30 is formed by adsorbing a plating catalyst on the catalyst adsorption layer 20 (FIG. 1C).

The plating catalyst is not particularly limited as long as it is a catalyst for electroless plating. For example, it is possible to use palladium (Pd), silver (Ag), copper (Cu), gold (Au), or platinum (Pt).

The formation of the catalyst layer 30 is performed by bringing a solution containing the plating catalyst into contact with the surface of the catalyst adsorption layer 20. The contact time of the surface of the catalyst adsorption layer 20 and the solution containing the plating catalyst is, for example, 1 minute or less.

Next, a metal film 40 is formed on the catalyst layer 30 by an electroless plating method (FIG. 1D). In FIG. 1D, the catalyst adsorption layer 20 and the catalyst layer 30 are not illustrated.

The metal film 40 is conformally formed between the protrusions 102 and on the protrusions 102. In other words, the metal film 40 is isotropically formed on the catalyst layer 30 between the protrusions 102 and on the protrusions 102 at substantially the same growth rate. The metal film 40 is buried between the protrusions 102.

The material of the metal film 40 is, for example, nickel (Ni), copper (Cu), cobalt (Co), or silver (Ag).

The formation of the metal film 40 is performed by dipping the substrate 100 into a plating solution. The plating solution contains, for example, a metal ion for forming the metal film 40, a reducing agent, and a stabilizer for stabilizing the metal ion. The dipping time of the substrate 100 into the plating solution is, for example, 2 minutes or less.

Next, the metal film 40 on the protrusions 102 is removed (FIG. 1E). The metal film 40 on the protrusions 102 is removed, and thus the metal film 40 is separated into a plurality of regions sandwiched between the protrusions 102.

The removal of the metal film 40 can be performed by, for example, publicly known wet etching. In addition, the removal of the metal film 40 can be performed by, for example, publicly known dry etching or a chemical mechanical polishing (CMP) method.

The separated metal film 40 can be used as a metal wiring of a semiconductor device.

Next, the function and effect of this embodiment will be described.

Along with the scaling-down of a semiconductor device, the scaling-down of a metal wiring is also required. In the case of forming a fine metal wiring using an electroless plating method, it is desired that a conformal metal film can be formed on a substrate having a fine uneven pattern on its surface. It is difficult to conformally form a metal film on a fine uneven pattern. In particular, when different materials exist on the surface, it is more difficult to form a conformal metal film by an electroless plating method which is easily affected by a base material.

In this embodiment, when forming the catalyst adsorption layer 20, a solution containing a triazine compound having a triazine skeleton, a first functional group of any one of a silanol group and an alkoxysilyl group, and a second functional group of at least one selected from the group consisting of an amino group, a thiol group, and an azido group is used. The triazine compound is represented by, for example,

Formula (1) below.

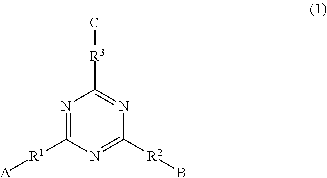

In Formula (1), at least one of A, B, and C is any one of a silanol group and an alkoxysilyl group, at least one of A, B, and C is at least one selected from the group consisting of an amino group, a thiol group, and an azido group, and R¹, R² and R³ are arbitrarily present linking groups. The triazine compound has a functional group of any one of at least one silanol group and an alkoxysilyl group at its terminal. Also, the triazine compound has at least one amino group, thiol group, or azido group at its terminal.

When the triazine compound of Formula (1) is used, it is possible to conformally form the metal film 40 in a fine uneven pattern where different materials exist on the surface. The reason for this is presumed that the dependence on the base material in the formation of the catalyst adsorption layer 20 is suppressed by using the triazine compound of Formula (1). Even when the aspect ratio of the height of the protrusion 102 to the interval between the protrusions 102 is, for example, 0.5 or more, it is possible to bury the metal film 40 between the protrusions 102. Even when this aspect ratio is, for example, 2 or more, it is possible to bury the metal film 40 between the protrusions 102.

Therefore, when the pitch of a wiring is, for example, 100 nm or less, it is possible to form an extremely fine metal wring by using an electroless plating method. Also, even when the pitch of a wiring pitch becomes small, it is possible to form a thick metal wiring. Thus, it is possible to form a low-resistance metal wiring even if scaling-down is performed.

Further, when the triazine compound of Formula (1) is used, it is possible to perform the formation of the catalyst adsorption layer 20 in a short time of, for example, 1 minute or less. Therefore, it is possible to form a metal wiring with high throughput.

Although a case where the protrusions 102 are formed of one layer of the silicon oxide layer 12 has been described as an example, for example, the protrusions 102 may have a structure in which two or more layers of different materials are stacked.

As described above, according to the method for forming a metal pattern according to this embodiment, it is possible to conformally form the metal film 40 on a substrate having a fine uneven pattern where different materials exist on its surface. Therefore, it is possible to form a fine and low-resistance metal wiring. Further, it is possible to form a metal wiring with high throughput.

### Second Embodiment

The method of forming a metal pattern according to this embodiment is different from that of the first embodiment in that the first material is an oxide, a nitride, or an oxynitride, and the second material is a resin. Hereinafter, a description of contents overlapping the first embodiment will not be repeated.

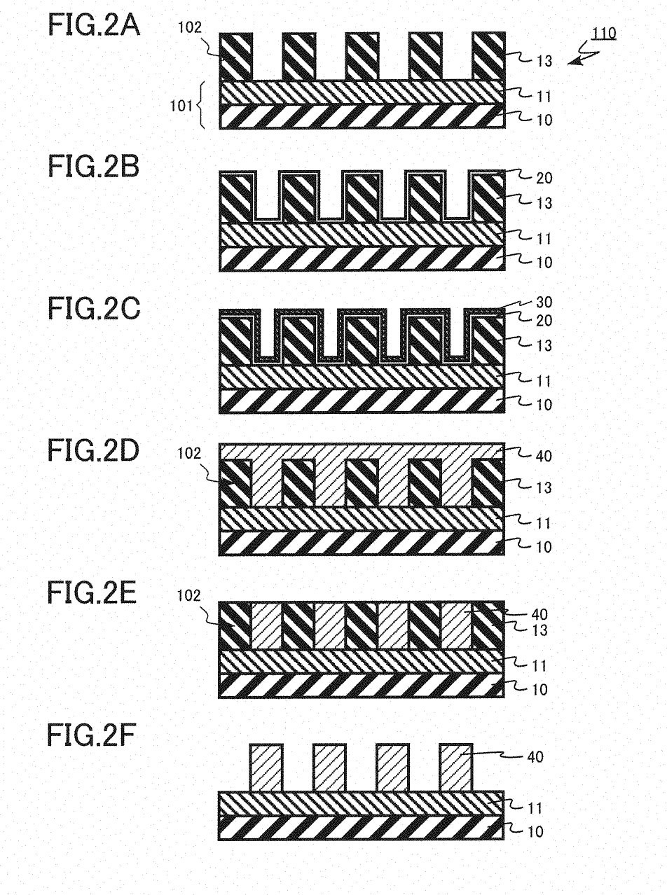

FIGS. 2A, 2B, 2C, 2D, 2E, and 2F are explanatory views of the method of forming a metal pattern according to this embodiment. FIGS. 2A, 2B, 2C, 2D, 2E, and 2F illustrate sectional views of a substrate on which a metal pattern is formed.

First, a substrate 110 is prepared (FIG. 2A). The substrate 110 is formed using a publicly known process technology.

The substrate 110 has a base region 101 and a plurality of protrusions 102. Further, the substrate 110 has a first material and a second material different from the first material. The first material and the second material are exposed on the surface of the substrate 110.

The first material is an oxide, a nitride, an oxynitride, or carbon. The second material is a resin. The oxide is, for example, silicon oxide or aluminum oxide. The oxide also includes SOG (Spin On Glass). When the first material is carbon, a carbon layer is formed by, for example, a coating method or a sputtering method. The nitride is, for example, silicon nitride or aluminum nitride. The oxynitride is, for example, silicon oxynitride or aluminum oxynitride. The resin is, for example, a photosensitive resin which is sensitive to light or an electron beam. The resin is, for example, a photoresist. Hereinafter, a case where the first material is silicon nitride and the second material is a photoresist will be described as an example.

The base region 101 includes a silicon layer 10 and a silicon nitride layer 11 on the silicon layer 10. A photoresist layer 13 is provided on the silicon nitride layer 11. The photoresist layer 13 is patterned to form a plurality of protrusions 102. Photoresist and silicon nitride are exposed on the surface of the substrate 110.

Next, the surface of the substrate 110 is brought into contact with a solution containing a triazine compound having a triazine skeleton, a first functional group of any one of a silanol group and alkoxysilyl group, and a second functional group of at least one selected from the group consisting of an amino group, a thiol group and an azido group, so as to form a catalyst adsorption layer 20 (FIG. 2B). The catalyst adsorption layer 20 is, for example, a monomolecular film.

Next, a catalyst layer 30 is formed on the catalyst adsorption layer 20. The catalyst layer 30 is formed by adsorbing a plating catalyst on the catalyst adsorption layer 20 (FIG. 2C).

Next, a metal film 40 is formed on the catalyst layer 30 by an electroless plating method (FIG. 2D). In FIG. 2D, the catalyst adsorption layer 20 and the catalyst layer 30 are not illustrated.

The metal film 40 is conformally formed between the protrusions 102 and on the protrusions 102. In other words, the metal film 40 is isotropically formed on the catalyst layer 30 between the protrusions 102 and on the protrusions 102 at substantially the same growth rate. The metal film 40 is buried between the protrusions 102.

Next, the metal film 40 on the protrusions 102 is removed (FIG. 2E). The metal film 40 on the protrusions 102 is removed, and thus the metal film 40 is separated into a plurality of regions sandwiched between the protrusions 102.

The removal of the metal film 40 can be performed by, for example, publicly known wet etching. In addition, the removal of the metal film 40 can be performed by, for example, publicly known dry etching or a CMP method.

Next, the photoresist layer 13 exposed between the metal films 40 is removed (FIG. 2F). The photoresist layer 13 can be removed by, for example, a publicly known ashing method.

The separated metal film 40 can be used as a metal wiring of a semiconductor device.

As the solvent of the solution containing the triazine compound, a solvent not dissolving the photoresist is used.

From this viewpoint, the solvent of the solution containing the triazine compound is preferably water.

As described above, according to the method for forming a metal pattern according to this embodiment, as described in the first embodiment, it is possible to form a fine and low-resistance metal wiring. Further, it is possible to form a metal wiring with high throughput.

### Third Embodiment

The method of forming a metal pattern according to this embodiment is different from that of the second embodiment in that the first material is an oxide, a nitride, an oxynitride, or carbon, that the second material is a resin or carbon, and that a metal film is removed, and then protrusions are removed, so as to etch a base region using the metal film as a mask. Hereinafter, a description of contents overlapping the second embodiment will not be repeated.

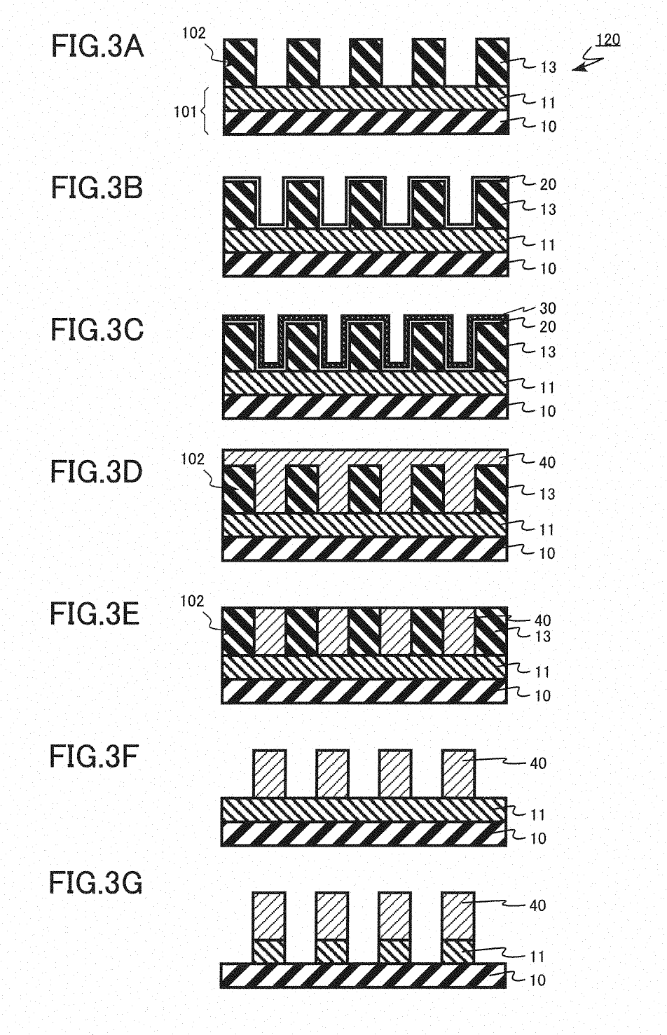

FIGS. 3A, 3B, 3C, 3D, 3E, 3F, and 3G are explanatory views of the method of forming a metal pattern according to this embodiment. FIGS. 3A, 3B, 3C, 3D, 3E, 3F, and 3G illustrate sectional views of a substrate on which a metal pattern is formed.

First, a substrate 120 is prepared (FIG. 3A). The substrate 120 is formed using a publicly known process technology.

The substrate 120 has a base region 101 and a plurality of protrusions 102. Further, the substrate 120 has a first material and a second material different from the first material. The first material and the second material are exposed on the surface of the substrate 120.

The first material is an oxide, a nitride, an oxynitride, or carbon. The second material is a resin or carbon. The oxide is, for example, silicon oxide or aluminum oxide. The oxide also includes SOG (Spin On Glass). The nitride is, for example, silicon nitride or aluminum nitride. The oxynitride is, for example, silicon oxynitride or aluminum oxynitride. The resin is, for example, a photosensitive resin which is sensitive to light or an electron beam. The resin is, for example, a photoresist. When the first material or the second material is carbon, a carbon layer is formed by, for example, a coating method or a sputtering method.

The protrusions 102 contain the second material, and the base region 101 contains the first material. Hereinafter, a case where the first material is silicon nitride and the second material is a photoresist will be described as an example.

The base region 101 includes a silicon layer 10 and a silicon nitride layer 11 on the silicon layer 10. A photoresist layer 13 is provided on the silicon nitride layer 11. The photoresist layer 13 is patterned to form a plurality of protrusions 102. Both photoresist and silicon nitride are exposed on the surface of the substrate 120.

Processes up to FIGS. 3B, 3C, 3D, 3E and 3F are the same as those in FIGS. 2B, 2C, 2D, 2E and 2F. That is, until the photoresist layer 13 exposed between the metal films 40 is removed (FIG. 3F), these processes are the same as those in the second embodiment.

Next, the silicon nitride layer 11 is etched using the separated metal film 40 as a mask (FIG. 3G). The silicon nitride layer 11 of the base region is patterned using the metal film 40 as a hard mask.

For example, when attempting to pattern a thick insulating layer into a fine pattern, there is a case where it is difficult to form a pattern if using a photoresist as a mask. This is caused by the fact that a sufficient etching selection ratio cannot be obtained between the photoresist and the insulating layer. Therefore, there is a method of using a metal, having a higher etching selection ratio with an insulating layer than a photoresist, as a mask, instead of a photoresist. This mask is referred to as a hard mask.

After the silicon nitride layer 11 is etched, the silicon layer 10, which is a lower layer, may be further etched. This embodiment can also be applied to a process of etching a plurality of layers on a substrate 120 having a base region of a multilayer structure using a metal as a hard mask.

In this embodiment, it is possible to form a fine and thick metal mask. Therefore, for example, even in the case of a thick insulating layer, it becomes possible to form a fine pattern by etching.

### Fourth Embodiment

The method of forming a metal pattern according to this embodiment includes: forming a catalyst adsorption layer by bringing a surface of a substrate into contact with a solution, the substrate having a base region and a photoresist layer provided on the base region, the photoresist layer having a plurality of protrusions, and the solution containing a compound having a triazine skeleton, a first functional group of any one of a silanol group and an alkoxysilyl group, and a second functional group of at least one selected from the group consisting of an amino group, a thiol group and an azido group; forming a catalyst layer on the catalyst adsorption layer; forming a metal film on the catalyst layer by an electroless plating method; removing the metal film on the protrusions; removing the photoresist layer between the metal films; and etching the base region using the metal film as a mask. This embodiment is different from the third embodiment in that a photoresist layer is used, and that a surface of a substrate is made of a single material. Hereinafter, a description of contents overlapping the third embodiment will not be repeated.

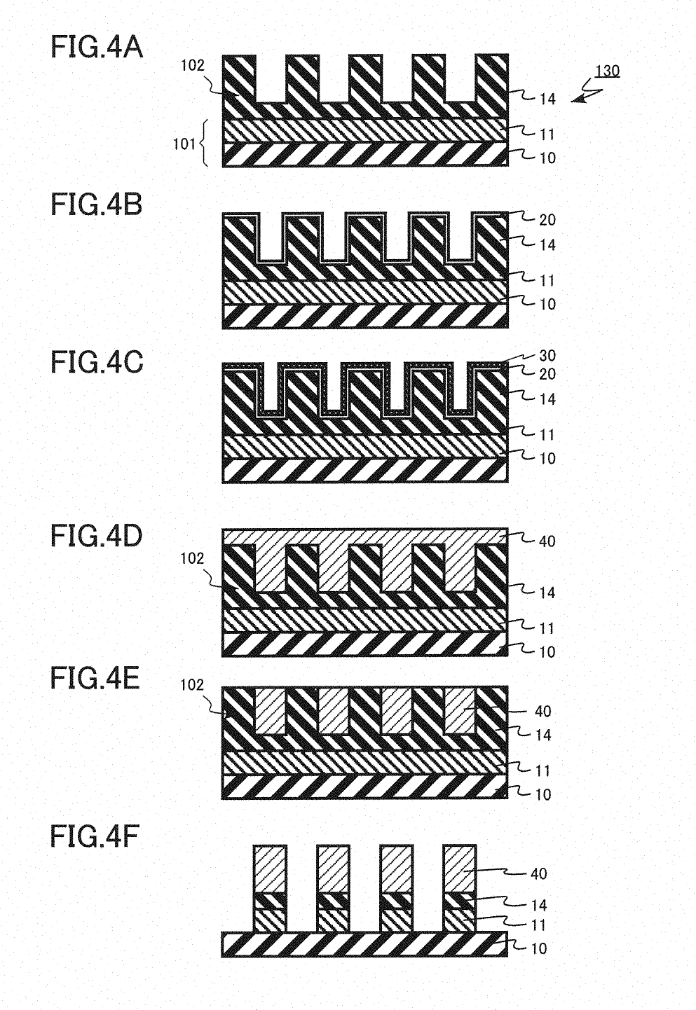

FIGS. 4A, 4B, 4C, 4D, 4E, and 4F are explanatory views of the method of forming a metal pattern according to this embodiment. FIGS. 4A, 4B, 4C, 4D, 4E, and 4F illustrate sectional views of a substrate on which a metal pattern is formed.

First, a substrate 130 is prepared (FIG. 4A). The substrate 130 is formed using a publicly known process technology.

The substrate 130 has abase region 101 and a plurality of protrusions 102. The plurality of protrusions 102 are formed on the surface of a photoresist layer. The photoresist layer is exposed on the surface of the substrate 130.

The photoresist is, for example, a photocurable resist for nanoimprinting, which is cured by irradiation with ultraviolet rays. Hereinafter, a case where the photoresist is a photocurable resist will be described as an example.

The base region 101 includes a silicon layer 10 and a silicon nitride layer 11 on the silicon layer 10. A photocurable resist layer 14 is provided on the silicon nitride layer 11. The photocurable resist layer 14 is patterned to form a plurality of protrusions 102. Photoresist and silicon nitride are exposed on the surface of the substrate 130.

Next, the surface of the substrate 130 is brought into contact with a solution containing a triazine compound having a triazine skeleton, a first functional group of any one of a silanol group and alkoxysilyl group, and a second functional group of at least one selected from the group consisting of an amino group, a thiol group and an azido group, so as to form a catalyst adsorption layer 20 (FIG. 4B).

Next, a catalyst layer 30 is formed on the catalyst adsorption layer 20. The catalyst layer 30 is formed by adsorbing a plating catalyst on the catalyst adsorption layer 20 (FIG. 4C).

Next, a metal film 40 is formed on the catalyst layer 30 by an electroless plating method (FIG. 4D). In FIG. 4D, the catalyst adsorption layer 20 and the catalyst layer 30 are not illustrated.

The metal film 40 is conformally formed between the protrusions 102 and on the protrusions 102. In other words, the metal film 40 is isotropically formed on the catalyst layer 30 between the protrusions 102 and on the protrusions 102 at substantially the same growth rate. The metal film 40 is buried between the protrusions 102.

Next, the metal film 40 on the protrusions 102 is removed (FIG. 4E). The metal film 40 on the protrusions 102 is removed, and thus the metal film 40 is separated into a plurality of regions sandwiched between the protrusions 102.

The removal of the metal film 40 can be performed by, for example, publicly known wet etching or dry etching.

Next, the photocurable resist layer 14 exposed between the metal films 40 is removed, and the silicon nitride layer 11 is etched using the separated metal film 40 as a mask (FIG. 4F). The removal of the photocurable resist layer 14 and the silicon nitride layer 11 can be performed by, for example, publicly known dry etching.

As the solvent of the solution containing the triazine compound, a solvent not dissolving the photocurable resist layer 14 is used. From this viewpoint, the solvent of the solution containing the triazine compound is preferably water.

As described above, according to the method for forming a metal pattern according to this embodiment, as described in the third embodiment, it is possible to form a fine and thick metal mask. Therefore, for example, even in the case of a thick insulating layer, it becomes possible to form a fine pattern by etching.

### Fifth Embodiment

The method of forming a metal pattern according to this embodiment includes: forming a catalyst adsorption layer by bringing a surface of a substrate into contact with a solution, the substrate including an insulating layer having a plurality of protrusions and a first metal film containing a first metal and provided on the insulating layer, and the solution containing a compound having a triazine skeleton, a first functional group of any one of a silanol group and an alkoxysilyl group, and a second functional group of at least one selected from the group consisting of an amino group, a thiol group and an azido group; forming a catalyst layer on the catalyst adsorption layer; forming a second metal film containing a second metal different from the first metal on the catalyst layer by an electroless plating method; and removing the first metal film and the second metal film on the protrusions after forming the second metal film. This embodiment is different from the first embodiment in that a second metal film is formed on a first metal film, and that a surface of a substrate is made of a single material. Hereinafter, a description of contents overlapping the first embodiment will not be repeated.

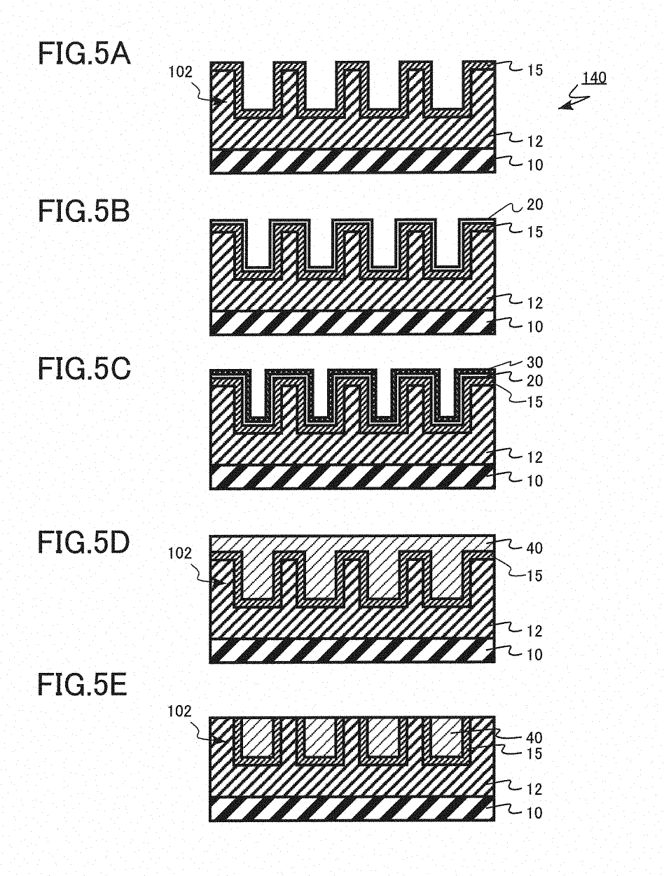

FIGS. 5A, 5B, 5C, 5D, and 5E are explanatory views of the method of forming a metal pattern according to this embodiment. FIGS. 5A, 5B, 5C, 5D, and 5E illustrate sectional views of a substrate on which a metal pattern is formed.

First, a substrate 140 is prepared (FIG. 5A). The substrate 140 is formed using a publicly known process technology. The substrate 140 has an insulating layer and a plurality of protrusions 102 provided on the insulating layer. A first metal film containing a first metal is formed on the insulating layer. The surface of the substrate 140 is the first metal film.

The insulating layer is made of, for example, an oxide, a nitride, or an oxynitride. The first metal is, for example, titanium (Ti), tungsten (W), or tantalum (Ta). The first metal film is, for example, a titanium layer, a titanium nitride layer, a tungsten nitride layer, or a tantalum nitride layer. Hereinafter, a case where the insulating layer is a silicon oxide layer will be described as an example.

The substrate 140 includes a silicon layer 10 and a silicon oxide layer 12 on the silicon layer 10. A plurality of protrusions 102 is formed on the silicon oxide layer 12. A first metal film 15 is formed on the silicon oxide layer 12. The first metal film 15 functions as a barrier metal of a metal wiring.

Next, the surface of the substrate 140 is brought into contact with a solution containing a triazine compound having a triazine skeleton, a first functional group of any one of a silanol group and alkoxysilyl group, and a second functional group of at least one selected from the group consisting of an amino group, a thiol group and an azido group, so as to form a catalyst adsorption layer 20 (FIG. 5B).

Next, a catalyst layer 30 is formed on the catalyst adsorption layer 20. The catalyst layer 30 is formed by adsorbing a plating catalyst on the catalyst adsorption layer 20 (FIG. 5C).

Next, a second metal film 40 containing a second metal is formed on the catalyst layer 30 by an electroless plating method (FIG. 5D). In FIG. 5D, the catalyst adsorption layer 20 and the catalyst layer 30 are not illustrated.

The second metal film 40 is conformally formed between the protrusions 102 and on the protrusions 102. In other words, the second metal film 40 is isotropically formed on the catalyst layer 30 between the protrusions 102 and on the protrusions 102 at substantially the same growth rate. The second metal film 40 is buried between the protrusions 102.

The second metal is, for example, nickel, copper, cobalt or silver. The second metal film 40 is, for example, a nickel layer, a copper layer, or a silver layer.

Next, the second metal film 40 on the protrusions 102 is removed (FIG. 5E). The second metal film 40 on the protrusions 102 is removed, and thus the second metal film 40 is separated into a plurality of regions sandwiched between the protrusions 102.

The removal of the second metal film 40 can be performed by, for example, publicly known wet etching. In addition, the removal of the second metal film 40 can be performed by, for example, publicly known dry etching or a CMP method.

The separated second metal film 40 can be used as a metal wiring of a semiconductor device.

The first metal film 15 functions as a barrier metal. The first metal film 15 suppresses, for example, the second metal film 40 from reacting with a base layer. Further, for example, the first metal film 15 suppresses the diffusion of the second metal in the second metal film 40 into the base layer.

As described above, according to the method for forming a metal pattern according to this embodiment, as described in the first embodiment, it is possible to form a fine and low-resistance metal wiring. Further, it is possible to form a metal wiring with high throughput. Moreover, it is possible to form a metal wiring containing a barrier metal.

### EXAMPLES

Hereinafter, Examples and Comparative Example will be described.

### Example 1

A substrate provided with a first silicon oxide layer, a silicon nitride layer, and a second silicon oxide layer was prepared. The silicon nitride layer and the second silicon oxide layer were etched to form an uneven pattern having a half pitch of 90 nm.

Both silicon nitride and silicon oxide are exposed on the surface of the substrate. The lower portion of a protrusion is silicon nitride, and the upper portion of the protrusion and the portion between the protrusions are silicon oxide.

The substrate was dipped into a triazine compound aqueous solution having a concentration of 0.1% for 30 seconds, and was then rinsed with pure water for 15 seconds, so as to form a catalyst adsorption layer. The triazine compound aqueous solution contains a triazine compound represented by Formula (1) above.

A 1 wt % palladium chloride hydrochloric acid solution was dipped into a palladium solution diluted with a 1% aqueous solution for 30 seconds, and was then rinsed with pure water for 15 seconds, so as to form a metal catalyst layer.

Subsequently, an electroless plating process was performed at 62° C. for 80 seconds using a NiB solution of pH 6.5 in which sodium hypophosphite is used as a reducing agent, so as to form a nickel layer.

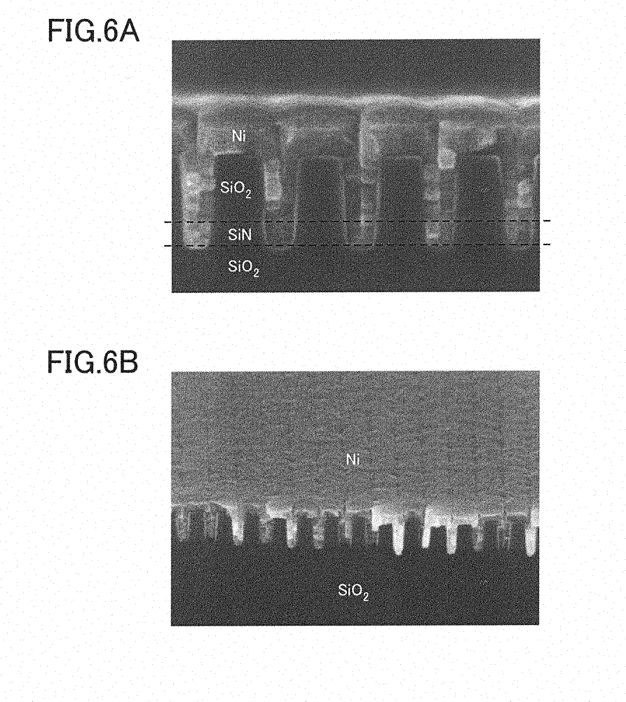

FIGS. 6A and 6B are SEM photographs of Example 1. FIG. 6A shows a sectional shape, and FIG. 6B shows a perspective shape.

As clearly shown in FIGS. 6A and 6B, a nickel layer is conformally formed on the fine uneven pattern.

### Comparative Example

A nickel layer was formed in the same manner as in Example 1, except that an organic aminosilane aqueous solution contains 3-aminopropyltrimethoxysilane having no triazine skeleton instead of the above triazine compound.

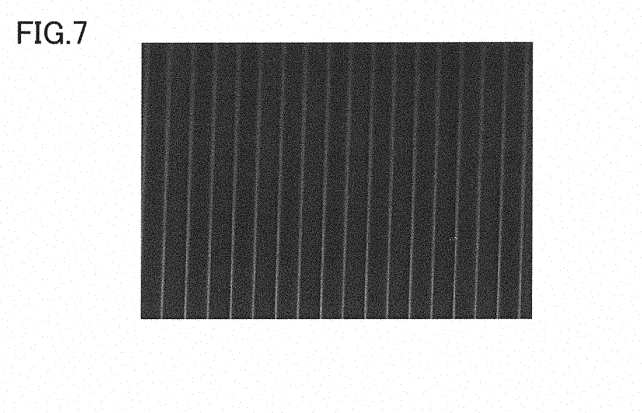

FIG. 7 is a SEM photograph of Comparative Example. FIG. 7 shows a top shape. From FIG. 7, it can be seen that no nickel layer was formed at all on a fine uneven pattern.

### Example 2

A substrate provided with a silicon layer, a silicon nitride layer, and a silicon oxide layer was prepared. The silicon nitride layer and the silicon oxide layer were etched to form an uneven pattern having a half pitch of 40 nm.

Silicon, silicon nitride, and silicon oxide are exposed on the surface of the substrate. The lower portion of a protrusion is silicon nitride, the upper portion of the protrusion is silicon oxide, and the portion between the protrusions is silicon.

A nickel layer was formed in the same manner as in Example 1 except that the substrate was different.

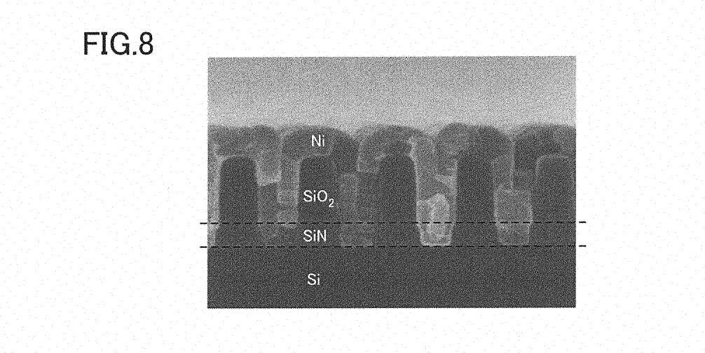

FIG. 8 is a SEM photograph of Example 2. FIG. 8 shows a sectional shape. As clearly shown in FIG. 8, a nickel layer is conformally formed on the fine uneven pattern.

### Example 3

A substrate provided with a silicon oxide layer and a photoresist layer was prepared. An uneven pattern having a half pitch of 40 nm was formed by the photoresist layer.

Silicon oxide and a photoresist are exposed on the surface of the substrate. A protrusion is a photoresist, and a portion between the protrusions is silicon oxide.

A nickel layer was formed in the same manner as in Example 1 except that the substrate was different.

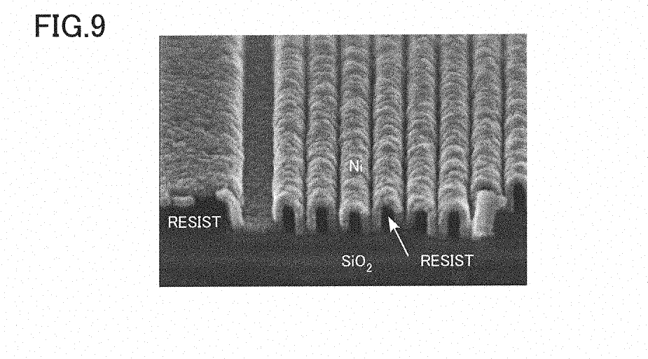

FIG. 9 is a SEM photograph of Example 3. FIG. 9 shows a perspective shape. As clearly shown in FIG. 9, a nickel layer is conformally formed on the fine uneven pattern.

### Example 4

A substrate provided with a silicon oxide layer and a nanoimprint resist layer was prepared. An uneven pattern having a half pitch of 30 nm was formed by the nanoimprint resist layer. The nanoimprint resist layer also exists between the protrusions.

A nanoimprint resist is exposed on the surface of the substrate. All surfaces between the protrusion and the protrusion are thermosetting resins.

A nickel layer was formed in the same manner as in Example 1 except that the substrate was different.

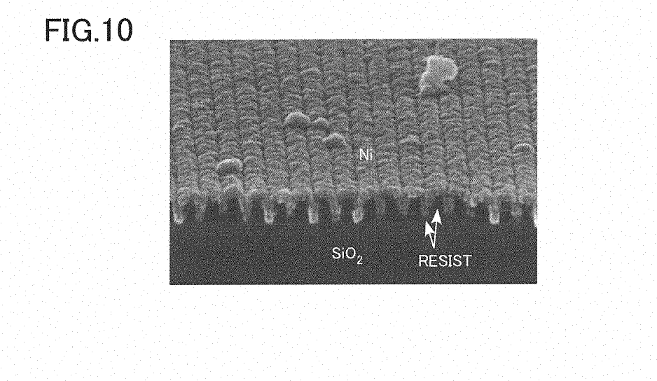

FIG. 10 is a SEM photograph of Example 4. FIG. 10 shows a perspective shape. As clearly shown in FIG. 10, a nickel layer is conformally formed on the fine uneven pattern.

### Example 5

A substrate provided with a carbon layer on which an uneven pattern having a half pitch of 40 nm was formed was prepared. The carbon layer also exists between the protrusions.

Carbon is exposed on the surface of the substrate. All surfaces between the protrusion and the protrusion are carbon.

A nickel layer was formed in the same manner as in Example 1 except that the substrate was different.

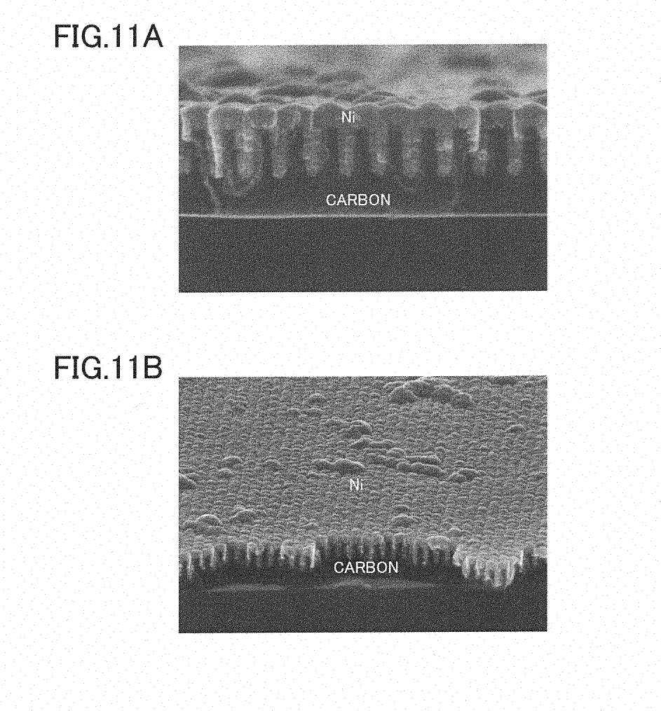

FIGS. 11A and 11B are SEM photographs of Example 5. FIG. 11A shows a sectional shape, and FIG. 11B shows a perspective shape. As clearly seen from FIGS. 11A and 11B, a nickel layer is conformally formed on the fine uneven pattern.

### Example 6

A substrate provided with a silicon oxide layer on which an uneven pattern having a half pitch of 40 nm was formed, and a barrier metal layer made of titanium nitride was prepared. Titanium nitride also exists between the protrusions.

Titanium nitride is exposed on the surface of the substrate. All surfaces between the protrusion and the protrusion are titanium nitride.

A nickel layer was formed in the same manner as in Example 1 except that the substrate was different.

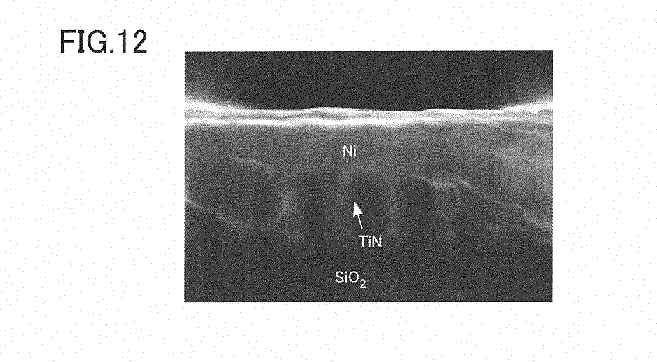

FIG. 12 is an SEM photograph of Example 6. FIG. 12 shows a sectional shape. As clearly seen from FIG. 12, a nickel layer is conformally formed on the fine uneven pattern.

In the first to fifth embodiments, a case where the disclosure is applied to the manufacture of a semiconductor device has been described as an example. However, the disclosure is not limited to the manufacture of a semiconductor device, and the disclosure can be applied other uses if a metal pattern is formed onto a substrate having an uneven pattern.

While certain embodiments have been described, these embodiments have been presented by way of example only, and are not intended to limit the scope of the inventions. Indeed, the method of forming a metal pattern described herein may be embodied in a variety of other forms; furthermore, various omissions, substitutions and changes in the form of the devices and methods described herein may be made without departing from the spirit of the inventions. The accompanying claims and their equivalents are intended to cover such forms or modifications as would fall within the scope and spirit of the inventions.

What is claimed is:

1. A method of forming a metal pattern, comprising:
forming a catalyst adsorption layer by bringing a surface of a substrate into contact with a solution, the substrate having a base region and a plurality of protrusions provided on the base region, the base region including a first material, the protrusions including a second material different from the first material, the first material and the second material being exposed on the surface of the substrate, and the solution containing a compound having a triazine skeleton, a first functional group of any one of a silanol group and an alkoxysilyl group, and a second functional group of at least one selected from the group consisting of an amino group, a thiol group, and an azido group;
forming a catalyst layer on the catalyst adsorption layer;
forming a metal film on the catalyst layer by an electroless plating method; and
removing the metal film on the protrusions.

2. The method according to claim 1, further comprising, after the removing the metal film, removing the protrusions, and etching the base region using the metal film as a mask.

3. The method according to claim 1, wherein the compound is a compound represented by Formula (1) below: 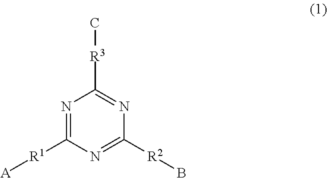
in Formula (1), at least one of A, B, and C is the first functional group, at least one of A, B, and C is the second functional group, and R¹, R² and R³ are arbitrarily present linking groups.

4. The method according to claim 1, wherein the first material and the second material are oxides, nitrides, or oxynitrides.

5. The method according to claim 1, wherein the first material is an oxide, a nitride, an oxynitride, or carbon, and the second material is a resin or carbon.

6. The method according to claim 1, wherein the second material is an oxide, a nitride, an oxynitride, or carbon, and the first material is a resin or carbon.

7. The method according to claim 1, wherein an arrangement pitch of the protrusions is 100 nm or less.

8. The method according to claim 1, wherein a ratio of a height of the protrusion to an interval between the protrusions is 0.5 or more.

9. A method of forming a metal pattern, comprising:
forming a catalyst adsorption layer by bringing a surface of a substrate into contact with a solution, the substrate having a base region and a photoresist layer provided on the base region, the photoresist layer having a plurality of protrusions, and the solution containing a compound having a triazine skeleton, a first functional group of any one of a silanol group and an alkoxysilyl group, and a second functional group of at least one selected from the group consisting of an amino group, a thiol group, and an azido group;
forming a catalyst layer on the catalyst adsorption layer;
forming a metal film on the catalyst layer by an electroless plating method;
removing the metal film on the protrusions;
removing the photoresist layer between the metal film; and
etching the base region using the metal film as a mask.

10. The method according to claim 9, wherein the compound is a compound represented by Formula (1) below: 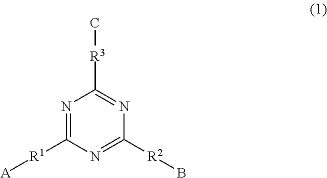
in Formula (1), at least one of A, B, and C is the first functional group, at least one of A, B, and C is the second functional group, and R¹, R² and R³ are arbitrarily present linking groups.

11. A method of forming a metal pattern, comprising:
forming a catalyst adsorption layer by bringing a surface of a substrate into contact with a solution, the substrate including an insulating layer having a plurality of protrusions and a first metal film containing a first metal and provided on the insulating layer, and the solution containing a compound having a triazine skeleton, a first functional group of any one of a silanol group and an alkoxysilyl group, and a second functional group of at least one selected from the group consisting of an amino group, a thiol group, and an azido group;
forming a catalyst layer on the catalyst adsorption layer;
forming a second metal film containing a second metal different from the first metal on the catalyst layer by an electroless plating method; and
removing the first metal film and the second metal film on the protrusions after forming the second metal film.

12. The method according to claim 11, wherein the compound is a compound represented by Formula (1) below: 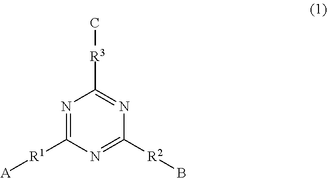
in Formula (1), at least one of A, B, and C is the first functional group, at least one of A, B, and C is the second functional group, and R¹, R² and R³ are arbitrarily present linking groups.
